(19) United States
(12) Patent Application Publication
(10) Pub. No.: US 2024/0016512 A1
(43) Pub. Date: Jan. 18, 2024

(54) Thrombectomy Apparatuses and Methods

(71) Applicant: Boston Scientific Scimed, Inc., Maple Grove, MN (US)

(72) Inventors: Robert J. Garabedian, Newark, CA (US); Teresa Ruvalcaba, San Leandro, CA (US)

(73) Assignee: Boston Scientific Scimed, Inc., Maple Grove, MN (US)

(21) Appl. No.: 18/220,999

(22) Filed: Jul. 12, 2023

Related U.S. Application Data
(60) Provisional application No. 63/388,569, filed on Jul. 12, 2022.

(51) Int. Cl. A61B 17/3207; A61B 17/22

(52) U.S. Cl. CPC A61B 17/32075; A61B 17/22031

(57) ABSTRACT

Medical devices and methods for using medical devices are disclosed. An example hand-held shaft puller for engaging a medical device shaft includes a handle having a distal end region, a proximal end region and a longitudinal axis. Further, the distal end region includes a first channel extending along the longitudinal axis, and wherein the first channel includes an engagement surface configured to engage an engagement member disposed on a shaft extending through the first channel.

### CROSS REFERENCE TO RELATED APPLICATIONS

This application claims the benefit of priority under 35 U.S.C. § 119 of U.S. Provisional Application No. 63/388,569, filed Jul. 12, 2022, the entire disclosure of which is hereby incorporated by reference and is related to U.S. Provisional Patent Application No. 63/151,054, titled “THROMBECTOMY TOOLS AND APPARATUSES” and filed on Feb. 18, 2021, and to U.S. Provisional Patent Application No. 63/249,561, titled “THROMBECTOMY TOOLS AND APPARATUSES” and filed on Sep. 28, 2021, each of which is herein incorporated by reference in its entirety.

### BACKGROUND

Removal of material from within a vessel or chamber is often useful. For example, removal of tissue such as blood clots from within a vasculature may improve patient conditions and quality of life. Clot removal may be beneficial or even necessary to improve patient outcomes. For example, in the peripheral vasculature, interventions and procedures can reduce the need for an amputation by 80 percent. The ultimate goal of any technique to treat these conditions is to remove the blockage and to restore patency, quickly, safely, and cost effectively.

Devices for mechanically removing material, including thrombus material, from with a lumen of the vessel may include an inverting tube for removing material from a body lumen, such as for removing a clot from a blood vessel (e.g., thrombectomy devices), are disclosed and described in in each of U.S. Pat. Nos. 10,271,864, 10,835,234, 10,561,431, 10,842,513 and 11,253,291. These apparatuses do an excellent job at removing material from within a blood vessel, but in some situations may face challenges when removing large amounts of material which may require multiple attempts to remove, and/or when removing softer clot material, which may be difficult to grip. In some cases, it may be difficult to completely remove clot material from against the wall of the vessel.

Thus, there is a need for devices, including thrombectomy devices, that can be remove tissue, and particularly large and/or soft materials, from within a body lumen. Described herein are apparatuses (devices, systems and kit) and methods of using them that may address the needs and problems discussed above.

### SUMMARY OF THE DISCLOSURE

This disclosure provides design, material, manufacturing method, and use alternatives for medical devices. An example hand-held shaft puller for engaging a medical device shaft includes a handle having a distal end region, a proximal end region and a longitudinal axis. Further, the distal end region includes a first channel extending along the longitudinal axis, and wherein the first channel includes an engagement surface configured to engage an engagement member disposed on a shaft extending through the first channel.

Alternatively or additionally to any of the embodiments above, wherein the proximal face is configured to engage an engagement member disposed along an inner shaft of a thrombectomy system.

Alternatively or additionally to any of the embodiments above, wherein the inner shaft includes a distal end region coupled to a flexible tube of the thrombectomy system.

Alternatively or additionally to any of the embodiments above, wherein the first channel includes a first diameter at a first longitudinal position along the first channel and a second diameter at a second longitudinal position along the first channel.

Alternatively or additionally to any of the embodiments above, wherein the handle is configured to be grasped by one hand.

Alternatively or additionally to any of the embodiments above, further comprising a second channel longitudinally aligned with the first channel.

Alternatively or additionally to any of the embodiments above, wherein the first channel and the second channel are longitudinally aligned to permit the inner shaft to extend along both the first channel and the second channel in a substantially straight configuration.

Alternatively or additionally to any of the embodiments above, wherein the engagement member includes a pull-ring.

Alternatively or additionally to any of the embodiments above, further comprising a slot positioned along the distal end region of the handle, wherein the slot is configured to accept an insert therein.

Alternatively or additionally to any of the embodiments above, wherein the insert is configured to engage the engagement member.

Alternatively or additionally to any of the embodiments above, wherein the slot extends through a lateral wall of the distal end region of the handle.

Alternatively or additionally to any of the embodiments above, wherein the handle includes a plurality of finger grips.

Another example hand-held shaft puller for engaging a shaft of a thrombectomy system includes a handle having a distal end region, a proximal end region and a longitudinal axis. Further, the distal end region includes a first channel extending along the longitudinal axis, the proximal end region includes a second channel longitudinally aligned with the first channel, the first channel includes a proximal face configured to engage a pull-ring disposed on an inner shaft extending through the first channel and a distal end region of the inner shaft is coupled to a proximal end of a flexible tube of the thrombectomy system.

Alternatively or additionally to any of the embodiments above, wherein the first channel and the second channel are longitudinally aligned to permit the inner shaft to extend along both the first channel and the second channel in a substantially straight configuration.

Alternatively or additionally to any of the embodiments above, further comprising a slot positioned along the distal end region of the handle, wherein the slot is configured to accept an insert therein.

Alternatively or additionally to any of the embodiments above, wherein the insert is configured to engage the pull-ring.

Alternatively or additionally to any of the embodiments above, wherein the slot extends through a lateral wall of the distal end region of the handle.

Alternatively or additionally to any of the embodiments above, wherein the distal end region of the handle includes a plurality of finger grips.

Alternatively or additionally to any of the embodiments above, wherein a distal end of the flexible tube is coupled to an outer shaft, and wherein the inner shaft is positioned within at least a portion of the outer shaft.

An example method of withdrawing an inner shaft of a thrombectomy system includes attaching a shaft puller to the inner shaft. The shaft puller includes a handle having a distal end region, a proximal end region and a longitudinal axis. Further, the distal end region includes a first channel extending along the longitudinal axis and the first channel includes a proximally-facing engagement surface. Additionally, the method includes engaging the engagement surface of the handle with a pull-ring disposed along the inner shaft and translating both the shaft puller and the inner shaft.

The above summary of some embodiments is not intended to describe each disclosed embodiment or every implementation of the present disclosure. The Figures and Detailed Description, which follow, more particularly exemplify these embodiments.

### BRIEF DESCRIPTION OF THE DRAWINGS

A better understanding of the features and advantages of the methods and apparatuses described herein will be obtained by reference to the following detailed description that sets forth illustrative examples, and the accompanying drawings of which:

FIGS. 1A-1C illustrate an example of an inverting tube (e.g., thrombectomy) apparatus that may be used to remove material from a vessel. In FIG. 1A, the assembled apparatus is shown in a side view, showing an inversion support catheter and a flexible outer tube. FIG. 1B shows the inverting tube apparatus of FIG. 1A in a vessel, proximal to a clot. FIG. 1C illustrates the removal of a clot from the vessel using the apparatus of FIG. 1A, by pulling the flexible tube on the outside of the inversion support catheter proximally so that it rolls over the distal end of the inversion support catheter and into the inversion support catheter, drawing the clot with it; the apparatus may be advanced distally.

FIGS. 2A-2B illustrate another example of an inverting tube apparatus including an expandable funnel that is attached to distal end of the inversion support catheter. FIG. 2B shows the inverting tube apparatus of FIG. 2A in a deployed configuration with an intermediate (e.g., delivery) catheter withdrawn proximally so that the expandable funnel at the distal end of the inversion support catheter can expand.

FIG. 3 illustrates a perspective view of an example shaft puller;

FIG. 4 illustrates a portion of the shaft puller of FIG. 3;

FIG. 5 illustrates another perspective view of the shaft puller of FIG. 3;

FIG. 6 illustrates a side view of the shaft puller of FIG. 3;

FIG. 7 illustrates another example shaft puller;

FIG. 8 illustrates a perspective view of the example shaft puller of FIG. 7;

FIG. 9 illustrates a side view of the shaft puller of FIG. 7.

### DETAILED DESCRIPTION

The methods and apparatuses described herein may also relate to improvement in the operation, and in particular, the insertion and use of, inverting tube apparatuses for removing material from within a body. These apparatuses may generally include an inversion support, which may include a catheter and in some examples a funnel region at the distal end of the catheter, a flexible tube configured to move over the outside of the inversion support and invert into the inversion support, and in some examples a puller attached to a first end of the inversion support for pulling the flexible tube into the inversion support. These apparatuses may be generally referred to as apparatuses for removing a material from a vessel and may be configured as mechanical thrombectomy apparatuses.

Also described herein are systems and methods for improving the ability of the flexible tube to grab material from the walls of the body lumen. Also described herein are methods for enhancing or improving the ability of the apparatus to grab clot by creating slack into the flexible tube before it inverts over the distal end of the inversion support catheter.

Any of these features, components and techniques may be used separately or in combination.

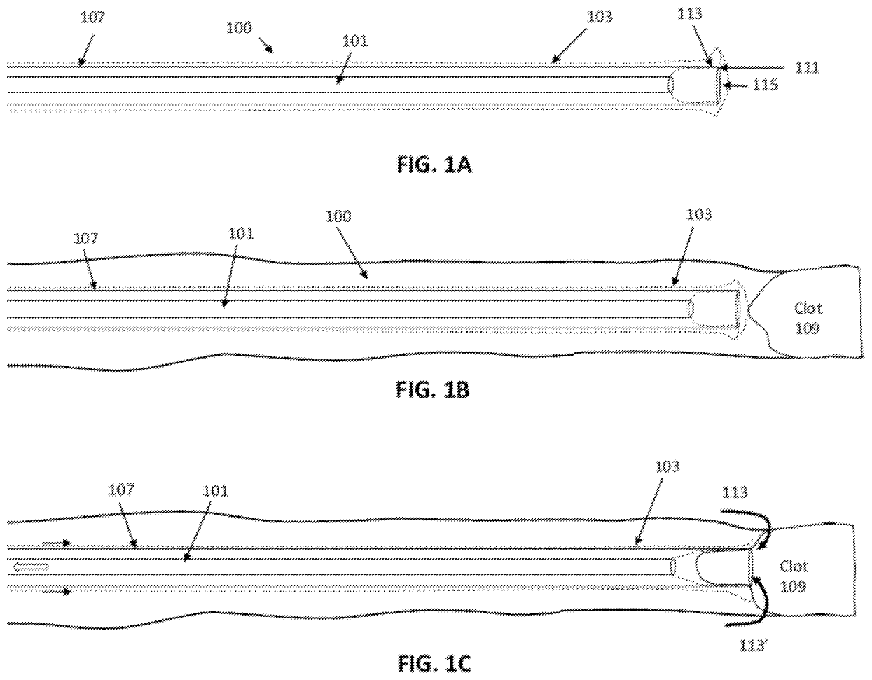

In general, an inverting tube apparatus (also referred to herein as “mechanical thrombectomy apparatus” or “inverting thrombectomy apparatus”) may be configured to remove material, such as clot, using a length of inverting tube, as shown in FIGS. 1A-1C. The apparatuses and methods of using them described herein may be used within the vasculature, including the neurovasculature and the peripheral vasculature.

For example, FIG. 1A illustrates an example of an inverting thrombectomy apparatus 100, such as described in U.S. Pat. No. 10,028,759, and in U.S. Pat. No. 9,463,035, the entirety of which are incorporated by reference. The apparatus includes an inversion support catheter 107 and a flexible tube 103 that extends over the outer surface of the inversion catheter 107. The flexible tube 103 may be referred to as a tractor tube (or flexible tractor tube) and may be attached at one end region to a puller 101, which may be a pull wire or a pull tube (e.g., catheter), e.g., at the distal end region of the puller 101. In some examples the flexible tube 103 may be attached proximal to the distal end of the puller 101 (e.g. between 1 mm and 50 mm from the distal end, between 1 mm and 40 mm, between 1 mm and 30 mm, greater than 5 mm, greater than 10 mm, greater than 20 mm, greater than 30 mm, etc. from the distal end of the puller). Pulling the puller 101 proximally inverts the flexible tube 103 over the distal end opening 111 of the inversion support catheter 107 to capture and remove a material (such as a clot) in the vessel lumen, as shown in FIGS. 1B and 1C. In operation, the amount of the material that may be captured corresponds to the length of the flexible tube 103.

In FIG. 1B the inverting tractor mechanical thrombectomy apparatus 100 is shown deployed near a clot 109. In the deployed configuration the puller 101 (shown here as a puller micro catheter, alternatively the puller 101 may be a wire) is held within an elongate inversion support catheter 107 so that the flexile tractor tube 103 extends from the end of the puller 101 and expands toward the inner radius of the elongate inversion support catheter 107. At the distal end opening 111 of the elongate inversion support catheter 107 the tractor tube 103 inverts over itself and extends proximally in an inverted configuration over the distal end of the elongate inversion support catheter 107. As shown in FIG. 1C, by pulling the puller 101 proximally, the tractor tube 103 rolls 113, 113′ and everts over the distal end opening of the elongate inversion support catheter 107, drawing the adjacent clot into the elongate inversion support catheter 107, as shown.

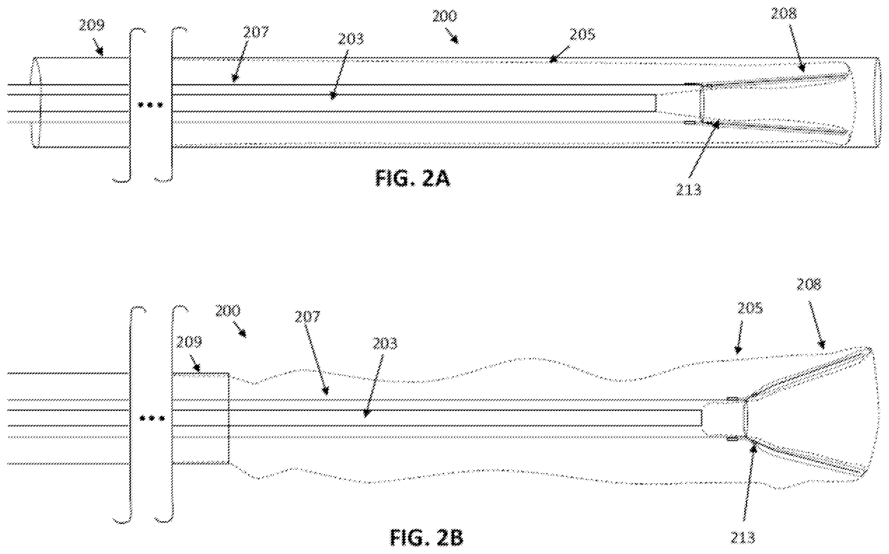

FIG. 1A the elongate inversion support catheter 107 is an elongate tube having a distal end that has the same size inner diameter as the proximal length of the inversion support catheter 107. In some examples the distal end of the inversion support catheter 107 may be funnel-shaped (or configured to expand into a funnel shape, see, e.g. FIGS. 2A-2B). In FIGS. 1A-1C, the inversion support catheter 107 is shown positioned between the tractor tube (e.g., flexible tube 103) and the puller 101 so that the flexible tube 103 can be pulled proximally by pulling on the puller 101 and rolling the flexible tube 103 into the elongate inversion support catheter 107 so that it inverts. The portion of the flexible tube 103 that is inverted over the distal end of the elongate inversion support catheter 107 has an outer diameter that is greater than the outer diameter of the elongate inversion support catheter 107. The flexible tube 103 may be biased so that it has a relaxed expanded configuration with a diameter that is greater than the outer diameter (OD) of the elongate inversion support catheter 107. In addition, the flexible tube 103 may also be configured (e.g., by heat setting, etc.) so that when the flexible tube 103 is everted and rolled over the distal end opening into the elongate inversion support catheter 107, the outer diameter of the flexible tube 103 within the elongate inversion support catheter 107 has an outer diameter that is about y times (y fold) the inner diameter of the elongate inversion support catheter 107 (e.g., where y is greater than 0.1×, 0.5×, 0.6×, 0.7×, 0.75×, 0.8×, 0.9×, 1×, etc. the inner diameter, ID, of the elongate inversion support catheter. This combination of an un-inverted diameter of the flexible tube 103 of greater than the diameter of the OD of the elongate inversion support catheter and an inverted diameter of the flexible tube of greater than, e.g., 0.7× the ID of the elongate inversion support catheter is surprisingly helpful for preventing jamming of the apparatus, both when deploying the apparatus and when rolling the flexible tube 103 over the distal end opening of the elongate inversion support catheter 107 to grab a clot. The flexible tube 103 may be expandable and may be coupled to the puller 101 as shown. In some examples the flexible tube 103 and the puller 101 may comprise the same material, but the flexible tube may be more flexible and/or expandable, or may be connected to elongate puller 101 (e.g., a push/pull wire or catheter). As mentioned above, the puller may be optional (e.g., the flexible tube may itself be pulled proximally into the inversion support catheter).

In FIG. 1C the clot may be drawn into the elongate inversion support catheter 107 by pulling the flexible tube 103 proximally into the distal end of the elongate inversion support catheter 107, as indicated by the arrows 113, 113′ showing pulling of the inner portion of the flexible tube 103, resulting in rolling the flexible tube 103 over the end opening of the catheter 107 and into the catheter distal end and inverting the expandable distal end region so that it is pulled into the catheter 107, shown by arrows. The end of the flexible tube outside of the catheter may be loose relative to the outer wall of the catheter.

2A-2B illustrate an example of an inverting tube apparatus that includes a funnel region at the distal end of an inversion support catheter. In this example the inverting tube apparatus 200 includes an elongate, flexible inversion support catheter 207 that has an expandable funnel 208 at the distal end, shown in a collapsed configuration in FIG. 2A within an intermediate (e.g., delivery) catheter 209, and in an open configuration in FIG. 2B after being released from the intermediate catheter. The funnel may be formed of a woven material and may be porous, particularly at the base region 213, where the funnel extends from the body of the elongate body of the inversion support catheter. A flexible tube 205 extends over the distal end (including the funnel) of the inversion support catheter and inverts over the distal opening of the funnel. The flexible tube may be, e.g., a knitted material, and may be biased to expand to an outer diameter (OD) that is larger than the OD of the funnel 208 in the open configuration. The flexible tube is attached to a distal end region of a puller 203. The flexible tube (e.g., “tractor”) is attached to the distal end region of the puller. The funnel may include two or more regions having different wall angles.

The apparatuses shown in FIGS. 1A-1C and 2A-2B may be modified or used with any of the methods and apparatuses described herein.

It can be appreciated that manipulating the a thrombectomy system, such as the thrombectomy system 100 illustrated in FIG. 1A-1, it may be necessary for a clinician to grasp the inversion support catheter 107 and the puller 101 to pull the puller 101 proximally (relative to the inversion support catheter 107) to evert the flexible tube 103 over the distal end opening of the elongate inversion support catheter 107, thereby drawing a clot into the elongate inversion support catheter 107 (as described herein).

Further, in some instances, a clinician may inadvertently apply excess force to the inversion support catheter 107, the puller 101 or both the inversion support catheter 107 and the puller 101 when performing a thrombectomy procedure. It can be appreciated that applying an excessive amount of force (e.g., grasping the shafts of the inversion support catheter 107 and/or the puller 101 with excessive force) may result in the inadvertent kinking, bending, warping, etc. of the inversion support catheter 107 and/or the puller 101. Accordingly, it may be desirable for a clinician to utilize a separate hand-held shaft pulling device to apply a sufficient withdrawal force on the puller 101 when withdrawing the puller 101 proximally with respect to the inversion support catheter 107. Specifically, it may be desirable for a clinician to use a hand-held shaft pulling device to attach to the puller 101 such that the shaft pulling device engages an engagement member attached to the puller 101 to facilitate withdrawing the puller 101 proximally (with respect to the inversion support catheter 107) without damaging (e.g., kinking) either the puller 101 or the inversion support catheter 107.

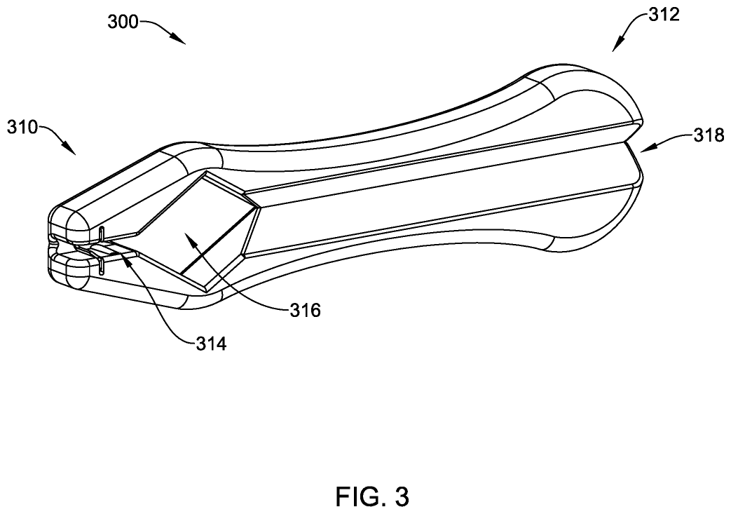

FIG. 3 illustrates an example shaft puller 300. The shaft puller 300 may include a distal end region 310 and a proximal end region 312. FIG. 3 further illustrates that the puller 300 may also include a first channel 314 positioned along the distal end region 310 of the puller 300. Additionally, the shaft puller 300 may include a second channel 318 that extends longitudinally from a medial region of the puller 300 to the proximal end region 312 of the puller 300. The first channel 314 may be longitudinally aligned with the second channel 318. Accordingly, it can be appreciated that both the first channel 314 and the second channel 318 may be configured to permit a shaft (e.g., the puller 101) to be disposed along both the first channel 314 and the second channel 318 such that the shaft (e.g., the puller 101) maintains a substantially straight (e.g., unkinked, unbent) configuration. Additionally, FIG. 3 illustrates that the puller 300 may include an aperture 316 disposed proximal to the distal end region 310 of the puller 300.

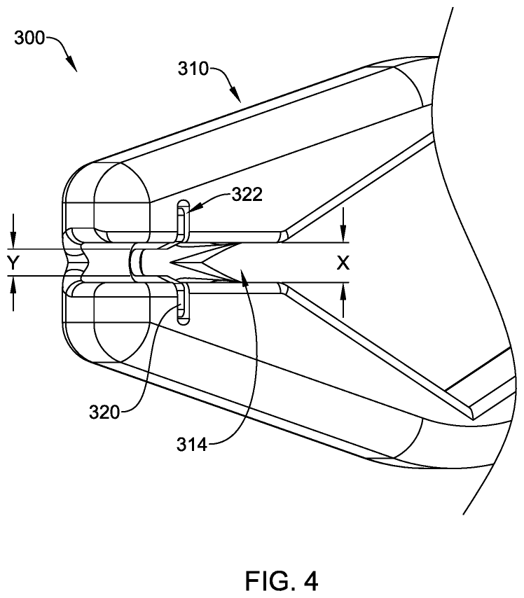

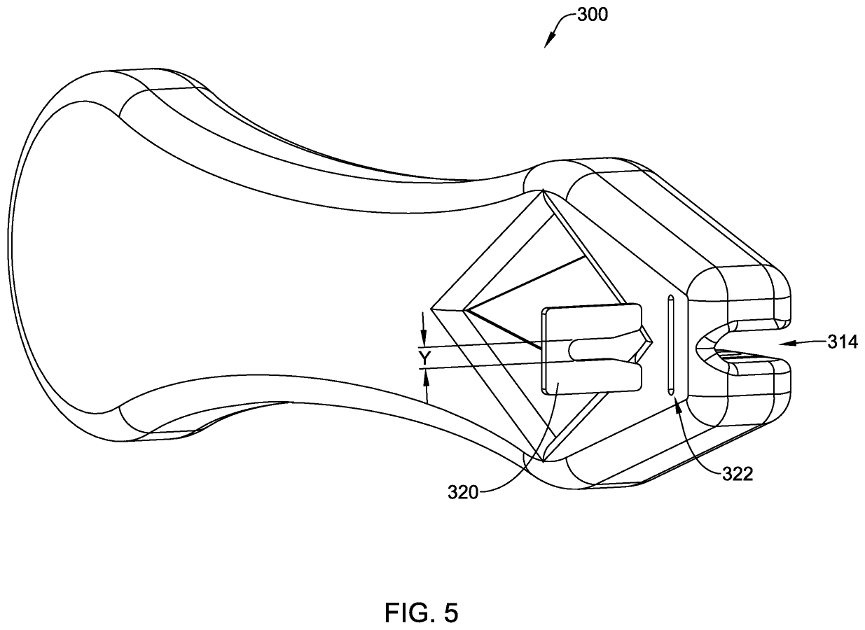

FIG. 4 illustrates a detailed view of the distal end region 310 of the shaft puller 300. FIG. 4 illustrates that the first channel 314 may have a diameter X. As discussed herein, the diameter X of the first channel 314 may be sized to permit a shaft (e.g., the puller 101) to be disposed therein. FIG. 4 further illustrates that the puller 300 may include a slot 322 extending laterally through the wall of the distal end region of the shaft puller 300 (FIG. 5 illustrates the opening of the slot 322 along a lateral side of the distal end region of the shaft puller 300).

FIG. 4 further illustrates that the shaft puller 300 may include an insert 320. The insert 320 may be configured to be disposed within the slot 322. For example, the insert 320 may include a profile which mates with the profile of the slot 322. FIG. 5 illustrates the insert 320 removed and spaced away from the slot 322.

As illustrated in FIGS. 4-5, the insert 320 may include an opening having a shape which substantially matches the shape of the first channel 314. However, the opening of the insert 320 may have a diameter Y, which is different than the diameter X of the channel 314, described above. For example, the diameter Y of the opening of the insert 320 may be less than the diameter X of the channel 314. Further, when inserted into the slot 322, the diameter X of the channel 314 may be reduced to the diameter Y at the longitudinal position where the insert 322 is inserted into the slot 322 along the length of the channel 314. It can be appreciated that the reduced diameter Y of the insert 320 may create a ridge (e.g., ledge, catch, lip, etc.) within the first channel 314.

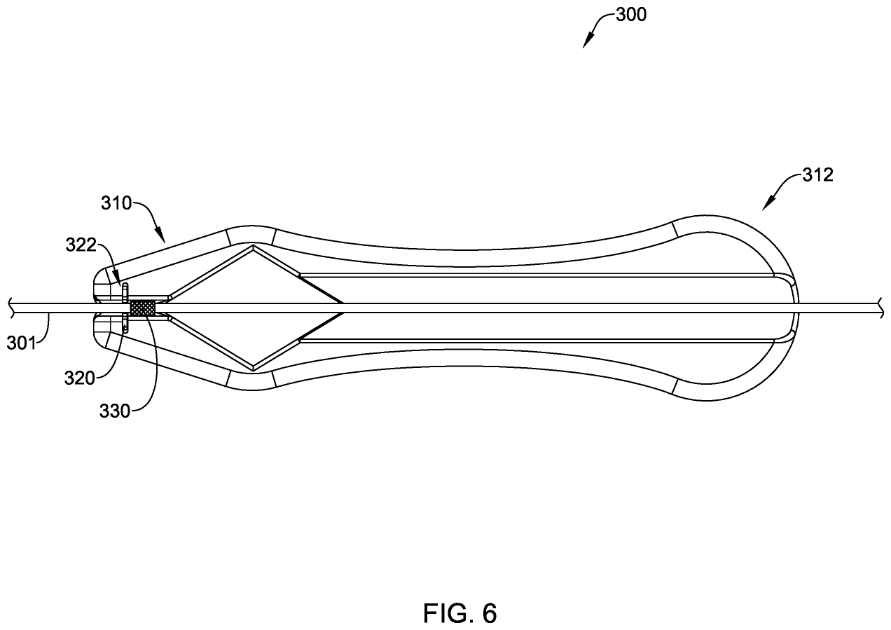

FIG. 6 illustrates a shaft 301 disposed within both the first channel 314 and the second channel 318 of the puller 300. The shaft 301 may be similar in form and function to the puller 101 described herein. FIG. 6 further illustrates that the shaft 301 may have a pull-ring 330 (e.g. collar) fixedly attached thereto. The pull-ring 330 may be attached to the shaft 301 using a variety of techniques including crimping, adhesive attachment, etc.

FIG. 6 further illustrates that the puller 300 has been positioned along the shaft 301 such that the pull-ring 330 is positioned proximal to the insert 320. Further, it can be appreciated that the pull-ring 330 may be configured to include an outer diameter that is less than the diameter X of the first channel 314 but greater than the diameter Y of the insert 320, thereby permitting the pull-ring 330 to be disposed within the channel 314 yet preventing the pull-ring 330 from advancing distally past the insert 320. Further yet, it can be appreciated that if the puller 300 is pulled in in a distal-to-proximal direction, the shaft 301 may pass through the first channel 314 until the insert 320 engages the pull-ring 330, thereby pulling the pull-ring 330, and consequently, the shaft 301, in a distal-to-proximal direction.

As discussed herein, the shaft 301 may represent the puller 101 described herein, and therefore, it can be appreciated that pulling the shaft 301 in a distal-to-proximal direction may represent a clinician using the puller 300 to pull the puller 101 in a distal-to-proximal direction to evert the flexible tube 103 over the distal end opening of the elongate inversion support catheter 107, and thereby drawing a clot into the elongate inversion support catheter 107.

It can be appreciated that the insert 320 may include openings have different diameters Y, which may permit the use of collars having diameters. For example, the puller 300 may include inserts 320 which permit the use of 3F to 8F diameter pull-rings.

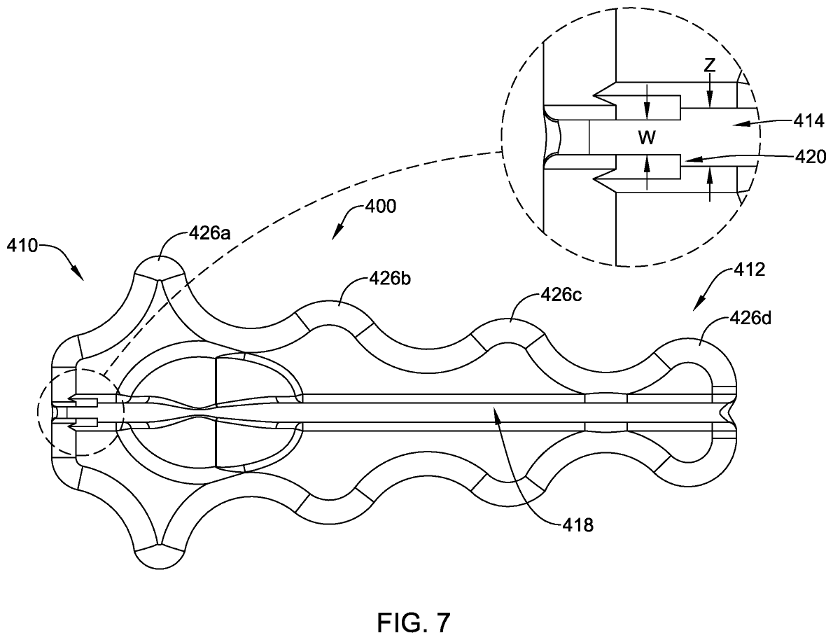

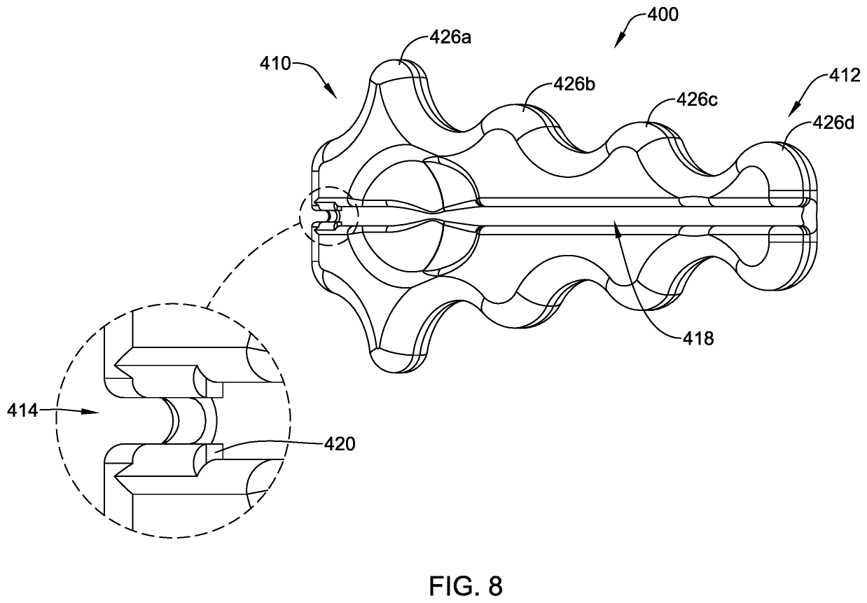

FIGS. 7-9 illustrates another example shaft puller 400. The shaft puller 400 may be similar in function to the puller 300 described above. For example, the shaft puller 400 may include a distal end region 410 and a proximal end region 412. Additionally, FIGS. 7-8 illustrate that the shaft puller 400 may include one or more ergonomic finger grips 426a, 426b, 426c, 426d positioned longitudinally along the body of the shaft puller 400.

FIG. 7 further illustrates that the puller 400 may also include a first channel 414 positioned along the distal end region 410 of the puller 400. Additionally, the shaft puller 400 may include a second channel 418 that extends longitudinally from a medial region of the puller 400 to the proximal end region 412 of the puller 400. The first channel 414 may be longitudinally aligned with the second channel 418. Accordingly, it can be appreciated that both the first channel 414 and the second channel 418 may be configured to permit a shaft (e.g., the puller 101) to be disposed along both the first channel 414 and the second channel 418 while maintaining a substantially straight (e.g., unkinked, unbent) configuration.

FIG. 7 further illustrates a detailed view of the distal end region 410 of the shaft puller 400. The detailed view of FIG. 7 illustrates that the first channel 414 may include a proximal channel region having a first diameter Z and a distal channel region having a diameter W. The diameter Z of the proximal channel region may be larger than the diameter W of the distal channel region of the first channel 414. However, it can be appreciated that both the distal channel region and the proximal channel region of the first channel 414 may be sized to permit a shaft (e.g., the puller 101) to be disposed therein. It can be appreciated that the reduced diameter W of the distal region of the channel 414 may create a proximal face 420 (e.g., ledge, face, ridge, catch, lip, etc.) within the first channel 414.

FIG. 8 illustrates a perspective view of the shaft puller 400. FIG. 8 illustrates the one or more ergonomic finger grips 426a, 426b, 426c, 426d extending along the shaft puller 400 from the distal end region 410 to the proximal end region 412. Additionally, FIG. 8 illustrates the proximal face 420 positioned within the first channel 414 of the distal end region 410 of the shaft puller 400.

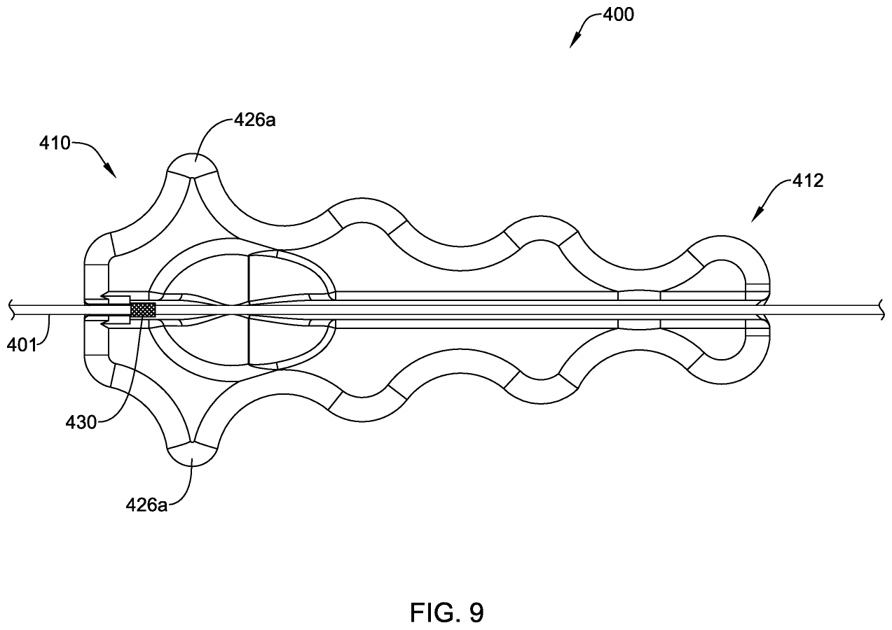

FIG. 9 illustrates a shaft 401 disposed within the first channel 414 and the second channel 418 of the puller 400. The shaft 401 may be similar in form and function to the puller 101 described herein. FIG. 9 further illustrates that the shaft 401 may have a pull-ring 430 (e.g., collar) fixedly attached thereto. The pull-ring 430 may be attached to the shaft 401 using a variety of attachment techniques including crimping, adhesive attachment, etc.

FIG. 9 further illustrates the puller 400 positioned along the shaft 401 such that the pull-ring 430 is positioned proximal to the proximal face 420. Further, it can be appreciated that the pull-ring 430 may be configured to include an outer diameter that is less than the diameter Z of the proximal channel region of the channel 414 but greater than the diameter W of the distal channel region of the channel 414, thereby permitting the pull-ring 430 to be disposed within the channel 414 yet preventing the pull-ring 430 from advancing distally past the proximal face 420. Further yet, it can be appreciated that if the puller 400 is pulled in in a distal-to-proximal direction, the shaft 401 may pass through the first channel 414 until the proximal face 420 engages the pull-ring 430, thereby pulling the pull-ring 430, and consequently, the shaft 401, in a distal-to-proximal direction.

As discussed herein, the shaft 401 may represent the puller 101 described herein, and therefore, it can be appreciated that pulling the shaft 401 in a distal-to-proximal direction may represent a clinician using the shaft puller 400 to pull the puller 101 in a distal-to-proximal direction to evert the flexible tube 103 over the distal end opening of the elongate inversion support catheter 107, and thereby drawing a clot into the elongate inversion support catheter 107.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative examples are described above, any of a number of changes may be made to various examples without departing from the scope of the invention as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the invention as it is set forth in the claims.

The examples and illustrations included herein show, by way of illustration and not of limitation, specific examples in which the subject matter may be practiced. As mentioned, other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is, in fact, disclosed. Thus, although specific examples have been illustrated and described herein, any arrangement calculated to achieve the same purpose may be substituted for the specific examples shown. This disclosure is intended to cover any and all adaptations or variations of various examples. Combinations of the above examples, and other examples not specifically described herein, will be apparent to those of skill in the art upon reviewing the above description.

What is claimed is:

1. A hand-held shaft puller for engaging a medical device shaft, comprising:
a handle having a distal end region, a proximal end region and a longitudinal axis;
wherein the distal end region includes a first channel extending along the longitudinal axis, and wherein the first channel includes an engagement surface configured to engage an engagement member disposed on a shaft extending through the first channel.

2. The shaft puller of claim 1, wherein the proximal face is configured to engage an engagement member disposed along an inner shaft of a thrombectomy system.

3. The shaft puller of claim 2, wherein the inner shaft includes a distal end region coupled to a flexible tube of the thrombectomy system.

4. The shaft puller of claim 1, wherein the first channel includes a first diameter at a first longitudinal position along the first channel and a second diameter at a second longitudinal position along the first channel.

5. The shaft puller of claim 1, wherein the handle is configured to be grasped by one hand.

6. The shaft puller of claim 1, further comprising a second channel longitudinally aligned with the first channel.

7. The shaft puller of claim 3, wherein the first channel and the second channel are longitudinally aligned to permit the inner shaft to extend along both the first channel and the second channel in a substantially straight configuration.

8. The shaft puller of claim 1, wherein the engagement member includes a pull-ring.

9. The shaft puller of claim 1, further comprising a slot positioned along the distal end region of the handle, wherein the slot is configured to accept an insert therein.

10. The shaft puller of claim 9, wherein the insert is configured to engage the engagement member.

11. The shaft puller of claim 10, wherein the slot extends through a lateral wall of the distal end region of the handle.

12. The shaft puller of claim 1, wherein the handle includes a plurality of finger grips.

13. A hand-held shaft puller for engaging a shaft of a thrombectomy system, comprising:
a handle having a distal end region, a proximal end region and a longitudinal axis;
wherein the distal end region includes a first channel extending along the longitudinal axis;
wherein the proximal end region includes a second channel longitudinally aligned with the first channel;
wherein the first channel includes a proximal face configured to engage a pull-ring disposed on an inner shaft extending through the first channel;
wherein a distal end region of the inner shaft is coupled to a proximal end of a flexible tube of the thrombectomy system.

14. The shaft puller of claim 13, wherein the first channel and the second channel are longitudinally aligned to permit the inner shaft to extend along both the first channel and the second channel in a substantially straight configuration.

15. The shaft puller of claim 13, further comprising a slot positioned along the distal end region of the handle, wherein the slot is configured to accept an insert therein.

16. The shaft puller of claim 15, wherein the insert is configured to engage the pull-ring.

17. The shaft puller of claim 16, wherein the slot extends through a lateral wall of the distal end region of the handle.

18. The shaft puller of claim 13, wherein the distal end region of the handle includes a plurality of finger grips.

19. The shaft puller of claim 13, wherein a distal end of the flexible tube is coupled to an outer shaft, and wherein the inner shaft is positioned within at least a portion of the outer shaft.

20. A method of withdrawing an inner shaft of a thrombectomy system, the method comprising:
attaching a shaft puller to the inner shaft, the shaft puller including:
a handle having a distal end region, a proximal end region and a longitudinal axis; wherein the distal end region includes a first channel extending along the longitudinal axis, and wherein the first channel includes a proximally-facing engagement surface; and
engaging the engagement surface of the handle with a pull-ring disposed along the inner shaft; and
translating both the shaft puller and the inner shaft.
